(19) United States
(12) Patent Application Publication
(10) Pub. No.: US 2022/0338506 A1
(43) Pub. Date: Oct. 27, 2022

(54) METHOD OF INCREASING THE PRODUCTIVITY OF A NON-RUMINANT ANIMAL

(71) Applicant: UNIVERSITY OF THE SUNSHINE COAST, Sippy Downs QLD (AU)

(72) Inventors: Nicholas PAUL, Sippy Downs QLD (AU); Valentin THEPOT, Sippy Downs QLD (AU); Alexandra CAMPBELL, Sippy Downs QLD (AU); Michael RIMMER, Sippy Downs QLD (AU)

(21) Appl. No.: 17/787,213

(22) PCT Filed: Dec. 10, 2020
(86) PCT No.: PCT/AU2020/051354

§ 371 Date: Jun. 17, 2022

(30) Foreign Application Priority Data
Dec. 19, 2019 (AU) 2019904825

(51) Int. Cl. A23K 10/30; A23K 50/80; A23K 50/75; A61K 36/04

(52) U.S. Cl. CPC A23K 10/30; A23K 50/80; A23K 50/75; A61K 36/04; A61K 2236/33

(57) ABSTRACT

The present invention relates to a method of increasing a measure of a production trait in a non-ruminant animal by administering a red seaweed of Asparagopsis species to the animal.

### TECHNICAL FIELD

The present invention relates to a method for increasing a measure of a production trait in a non-ruminant animal.

### BACKGROUND

Any references to methods, apparatus or documents of the prior art are not to be taken as constituting any evidence or admission that they formed, or form part of the common general knowledge.

Production traits are characteristics of animals, such as the quantity or quality of meat, fibre, eggs, growth rates, fertility, appetite stimulant and feed utilisation that they (or their progeny) produce, which contribute directly to the value of the animals for the farmer, and that are identifiable or measurable at the individual level. Production traits of farm animals are generally quantitatively inherited, i.e. they are influenced by many genes whose expression in a particular animal also reflects environmental influences. Two main opportunities are available to improve production in animals: through the modification of animal management to show increased measures of a production trait or by modifying the genetic characteristics of the animals to improve the production trait. However, animal management in non-ruminant animals is vastly different to animal management in ruminant animals due to their different physiology.

The digestive tract of ruminants contains four major parts; the abomasum, rumen, omasum and reticulum. The food is first chewed and mixes with saliva. The chewed food passes to the rumen for breaking them into smaller particles, and then it moves to the reticulum where the food is broken into further smaller particles.

Indigestible particles are sent back for rechewing and then to rumen. A group of Archaea known collectively as methanogens in the rumen assist in the breakdown of cellulose in the rumen but produce methane as a by-product of their metabolism. The partially digested food then passes from the rumen to the omasum which, decreases the pH level and thus initiates the release of enzymes for further break down the food, which is later passed to the abomasum that absorbs remaining nutrients before excretion. This process takes about 9-12 hours.

The digestive tract of non-ruminant animals is vastly different, and they have different protein requirements to ruminants (Angell et al. 2016). Non-ruminant animals have a simple stomach. Non-ruminant animals include fish and chicken. The structural components of a fish's digestive system include the mouth, teeth and gill rakers, oesophagus, stomach, pylorus, pyloric caeca, pancreatic tissue (exocrine and endocrine), liver, gall bladder, intestine and anus. In predatory (carnivorous) fishes, the mouth is usually large for engulfing prey whole, or in large chunks, and teeth are present on the jaws (e.g. maxillary and dentary) and tongue (e.g. glossyhyal) for grasping live prey. In omnivorous and planktivorous fishes, the mouth is smaller and is usually devoid of teeth except for pharyngeal teeth that may be blunt and flat (molariform) for grinding or sharp and long for shredding. Gill rakers in these fish are typically fine to prevent the escape across the gills of small food particles. In carnivorous fish, the stomach is muscular and elastic for holding large prey items, while in omnivorous and planktivorous fishes the stomach, if present at all, is small because a more or less constant stream of small food particles can flow directly into the intestine. Molluscs also have a simple digestive system. The mouth leads into a hard and round muscular pharynx or buccal mass, containing a pair of horny jaws that are moved by strong muscles to cut prey. An oesophagus leads from the buccal mass to a large, sac-like and thick walled muscular cliverticulum stomach where digestive fluids digest the food. The stomach is followed by a short intestine, demarcated by a constriction from the rectum, which terminates in the anus. Other shellfish such as prawns have a digestive tract which includes a stomach in which digestion takes place through the action of secreted digestive enzymes.

Chickens do not have teeth, and so digest their food differently to mammals. The beak moistens food with saliva, but it is not chewed. The oesophagus takes the food down to the crop to be stored. Food from the crop slowly passes down to the proventriculus. The proventriculus mixes the food with acids and digestive enzymes. Food is then passed through to the gizzard where insoluble (flint) grit has accumulated, where it is ground down by strong muscular action. From the gizzard, food is passed through to the small intestine for digestion and ultimate passage via the cloaca as a combination of faeces and urine

WO2018/018062 describes the utilisation of a red marine macroalgae, Asparagopsis taxiformis, to provide improvements in growth performance of ruminant animals for red meat production, especially cattle in pasture in feedlot farming systems. The red marine macro alga is admixed with animal feed components, mixed in with the feedlot ration or mixed with lick block components and moulded into a lick block. The specification indicates that the harvested biomass requires freezing as soon as possible to reduce loss of volatile bromoform compounds.

WO2015/109362 indicates that Asparagopsis inhibits methane production and uses the energy and carbon saved for formation of volatile fatty acids essential to ruminant nutrition. The methane produced by ruminant animals is belched or emitted to the atmosphere. In fact, sheep can produce about 30 L of methane each day and a dairy cow up to about 200 L. While fish are observed to gulp air to inflate their bladder to maintain buoyancy and expel the air through either their mouth or gills, they do not expel gases that are a by-product of digestion. Birds such as chickens extruded combination of faeces and urine through digestive systems vastly different to those of ruminant animals. Indeed, while the digestive system of ruminants relies on methanogens to break down cellulose (as it is their main source of nutrition) chickens are omnivores and fish are either carnivorous or consume plankton or algae.

In view of these differences, excess methane production is not a noted problem for non-ruminant animals. Therefore it would not be expected that consumption of the food containing chemical compounds which inhibit methane production by methanogens found in the rumen of ruminant animals would have any effect on non-ruminant animals such as fish and birds, nor that consumption of such a food would increase production in non-ruminant animals.

### SUMMARY OF INVENTION

It has now, surprisingly, been discovered that administering a red marine macroalgae to non-ruminant animals with simple digestive systems, can increase production in those animals.

In one aspect, the invention provides a method for increasing a measure of a production trait in a non-ruminant animal, comprising the step of administering to said animal a red seaweed of Asparagopsis species, an extract therefrom or the residue of the seaweed following an extraction process.

### BRIEF DESCRIPTION OF THE DRAWINGS

Preferred features, embodiments and variations of the invention may be discerned from the following Detailed Description, which provides sufficient information for those skilled in the art to perform the invention. The Detailed Description is not to be regarded as limiting the scope of the preceding Summary of the Invention in any way.

The Detailed Description will make reference to a number of drawings as follows:

FIG. 1 is a graph showing average fish weight (a) at the end of the 4 weeks trial and (b) at the 2 week point and (c) specific growth rate (SGR) at the end of 4 weeks and (d) the food conversion ratio (FCR) at the end of 4 weeks as described in Example 2. In the legend Whole designates whole seaweed; Extract is an extract of whole seaweed at doses equivalent to 1.5% inclusion, 3% inclusion and 6% inclusion of whole seaweed and Residue designates the residue after extraction as set forth in Table 2; and

FIG. 2 is a graph showing survival rate post stress test at the end of the 4 weeks trial described in Example 5 (rabbitfish). In the legend Whole designates whole seaweed; Extract is an extract of whole seaweed at doses equivalent to 1.5% inclusion, 3% inclusion and 6% inclusion of whole seaweed and Residue designates the residue after extraction as set forth in Table 2; and

FIG. 3a shows the relative abundance heat maps of the bacteria representing more than 1% of the total abundance (identified from order down to genus) in the gut of fish (rabbitfish) described in Example 2 and Example 4. FIG. 3b shows the relative abundance heat maps of the bacteria representing more than 1% of the total abundance (identified from order down to genus) in the gut of rabbitfish from Example 5 and FIG. 3c shows the alpha diversity (Shannon index) of the gut microbiome from fish fed Asparagopsis whole, extract and residue described in Example 5; and

FIG. 4 shows average fish weight (salmon) at the end of (a) the 4 weeks trial and (b) the 2 week point, and (c) the relative growth rate (%) at the end of 4 weeks trial described in Example 6. In the legend Control designates the control diet with no treatments; 3% Whole designates whole seaweed at 3% inclusion; Extract is an extract of whole seaweed at doses equivalent to 3% inclusion and equivalent to 6% inclusion of whole seaweed described in Example 7; and LPS designates lipopolysaccharide added as a positive control (0.01% inclusion, W/W); and

FIG. 5 shows fish feed consumption (salmon) after 2 weeks trial as (a) total feed consumed and (b) feed consumption as a proportion of biomass described in Example 6. In the legend Control designates the control diet with no treatments; Whole 3% designates whole seaweed at 3% inclusion; Extract is an extract of whole seaweed at doses equivalent to 3% inclusion and 6% inclusion of whole seaweed described in Example 7; and LPS designates lipopolysaccharide added as a positive control; and

FIG. 6a shows the relative abundance heat maps of the bacteria representing more than 1% of the total abundance (identified from order down to genus) in the gut microbiome from fish (salmon) fed Asparagopsis whole, extract and the positive control LPS described in Example 6. FIG. 6b shows the alpha diversity (Observed ASVs; [Amplicon sequence variants]) of the gut microbiome from fish fed Asparagopsis whole, extract and the positive control LPS described in Example 6. In the legend Control designates the control diet with no treatments; Whole 3% designates whole seaweed at 3% inclusion; Extract is an extract of whole seaweed at doses equivalent to 3% inclusion and 6% inclusion of whole seaweed described in Example 7; and LPS designates lipopolysaccharide added as a positive control; and

FIG. 7 is a graph showing the proportion of sexually mature individuals (rabbitfish) after feeding for three months described in Example 8. In the legend Control designates the control diet with no treatments; Whole 3% designates whole seaweed at 3% inclusion; Extract is an extract of whole seaweed at a dose equivalent to 3% inclusion of whole seaweed described in Example 7; and Hilyses designates the β-glucan rich commercial extract Hilyses® added as a positive control (3% inclusion, W/W).

### DETAILED DESCRIPTION OF PREFERRED EMBODIMENTS

Asparagopsis is a genus of red algae. It comprises two species, Asparagopsis armata and Asparagopsis taxiformis. Asparagopsis species are marine algae (‘seaweeds’). Asparagopsis armata is native to the Southern Hemisphere, having first been described in Western Australia in 1855. According to AlgaeBase (www.algaebase.org) Asparagopsis armata has been introduced into the Mediterranean Sea and is now frequent and widespread in the western Mediterranean. Like many red algae, its life cycle has two distinct phases that are very different in appearance (although the biochemistry is remarkably similar between the two phases e.g. Paul, et al., 2006; Verges, et al., 2008); the gametophyte phase and the tetrasporophyte phase. The gametophyte phase is most abundant in Australia and Europe between June and December. It is pale purple-red, and it has irregularly branched thalli that are typically 20 mm wide and up to 200 mm long. The lower branchlets of Asparagopsis armata have characteristic harpoon-like barbs, leading to the common name of “harpoon weed”. The tetrasporophyte phase (previously identified as Falkenbergia rufolanosa) occurs in Australia and Europe all year round. It is brownish red, branched and filamentous and grows in 1 mm diameter tufts.

Asparagopsis taxiformis is distributed in tropical/subtropical oceans from Rottnest Island, Western. Australia to southern Queensland. A. taxiformis does not have barbs. In Australia it is commonly referred to as “iodine” weed as it smells like an iodine tincture. The species has been introduced to the Mediterranean Sea by shipping. Asparagopsis taxiformis also has a haplodiplophasic lifecycle, with the haploid phase previously being identified as Falkenbergia hillebrandii.

For the avoidance of doubt, references to “Asparagopsis” generally, “Asparagopsis species”, “Asparagopsis spp.” or “Asparagopsis sp.” refers to all species in the genus Asparagopsis. Since taxonomic names can change, and species can be re-classified, the term also refers to species within the genus named using previous names and species within any future genus covering the organisms presently in the genus Asparagopsis.

In an embodiment the red seaweed is Asparagopsis taxiformis.

In an embodiment the red seaweed is Asparagopsis armata.

The present inventors have discovered that red seaweed of Asparagopsis species is effective in increasing a measure of a production trait in a non-ruminant animal.

Birds such as chicken are used for food, but also for egg production. Accordingly, traits such as egg production, egg weight, egg colour, shell strength, age at sexual maturity, body weight, albumen height, and yolk weight are important.

In an embodiment the non-ruminant animal is a bird. In an embodiment the bird is one of the poultry species. Poultry species are domesticated birds kept by humans for their eggs, their meat or their feathers. These birds are most typically members of the superorder Galloanserae, especially the order Galliformes. Poultry birds include chickens (including bantams), quails, turkeys, emus, fowls such peafowl and guinea fowl, swans, turkey, geese, ducks, ostriches, pheasants, partridges and pigeons.

Fish farms are complex production systems for two reasons. Fish are kept outdoors in most farming systems and, thus, are exposed to fluctuating environmental conditions. Seasonal variation in temperature causes variation in growth rate of fish, because of their ectothermic nature. Fish are harvested at a constant weight rather than at a constant age, hence the length of a production cycle depends on the stocking date and growth rate. Therefore, improvements in the growth rate of fish are important to boost productivity. Treatment of animals such as fish with red seaweed of Asparagopsis species is an alternative to other growth promotion practices such as administration of antibiotics in sub-therapeutic amounts to healthy animals. The person skilled in the art will appreciate that treatment of animals such as fish with red seaweed of Asparagopsis species may also be used in conjunction with other growth promotion practices.

In an embodiment the animal is one of the bony fishes, particularly one of the teleosts, or ray-finned fishes.

The present invention can be practiced with any of the considerable variety of fresh, brackish, or salt water fish species including, but not limited to: barramundi, catfish, carp, trout, salmon, tuna, cobia, char, whitefish, sturgeon, tench, roach, pike, pike-perch, sole, turbot, halibut, yellowtail, bass, bream, kingfish, milkfish, tilapia, mullet, grouper, eels and aquarium fish such as goldfish, angel fish, clown fish, cichlids, corydoras, danio, discus, eel, gourami, guppy, loach, minnow, molly, platy, Plecostumas, rainbow and platy variatus, rasbora, shark, sword, tetra, botia, knife fish, lionfish, archer fish, flounder, goby, half beak, mono, needle fish, pipe fish, puffer, scat (green and red), rabbitfish, bumble bee, twin spot damsel, yellowtail damsel, barbed squirrel, wrasse, black-spotted puffer, trigger fish, puffer, and butterfly fish. Yet other species with which the present invention can be practiced will be apparent to the person skilled in the art. The person skilled in the art will appreciate that commercial species in a particular country may be determined by availability locally, and also that they may be named differently in different locations.

In an embodiment the fish is a commercial species that is farmed. In particular, the fish is selected from the group consisting of salmon, tuna, trout, sea bass, turbot, halibut, sea bream, kingfish, barramundi, grouper, carp, tilapia, and catfish. In an embodiment the fish is salmon.

In an embodiment the animal is a type of shellfish, particularly one of the crustacea, or gastropod, echinoderm or annelid species.

In an embodiment the shellfish is a commercial species that is farmed. In particular, the shellfish is selected from the group consisting of prawns, shrimps, lobsters, crayfishes, yabbies, crabs, abalone, mussels, oysters, cockles, sea urchins, sea cucumbers and polychaete worms. In an embodiment the shellfish is a prawn or shrimp.

As used herein, the term “production traits” refers to characteristics of animals, such as the quantity or quality of meat, fibre, eggs, growth rates, fertility, appetite stimulant and feed utilisation that they (or their progeny) produce, which contribute directly to the value of the animals for the farmer, and that are identifiable or measurable at the individual level.

The red seaweed may be administered to the animal by any suitable method. The red seaweed can be administered in a solid form. This may be in the form of dried seaweed, which may be pulverized or powdered. The dried seaweed can be formulated as a veterinary formulation or as a feed supplement. In an embodiment it is physically mixed with feed material in a dry form. The red seaweed may also be formed into a liquid formulation and thereafter sprayed onto feed material. The red seaweed may be introduced to an animal in any suitable way. For example, it may be spread upon the water for fish to consume or added to drinking water or feed for a land animal.

Animals, including fish, will not generally eat the seaweed in its natural form as they do not find it palatable. Therefore, while the red seaweed may be fed directly to animals, it is preferable to formulate it into an animal feed to make it palatable. In embodiments where the red seaweed is fed directly to animals it would generally be collected and dried before doing so. Advantageously, it would be collected, dried and powdered before feeding to the animal. Typically, it would be collected, dried, powdered and pelletised before feeding to the fish. In an embodiment, the red seaweed would be pelletised along with other animal feed components. In an alternative embodiment, seaweed in powdered or pelletised form is used to supplement animal feed. A supplement may be mixed into an animal feed, administered separately at the same time as the animal feed, or administered at a different time to the animal feed provided that the animal will consume it.

The seaweed may be treated after harvest, for example, to concentrate bioactive compounds and/or to facilitate storage and/or to facilitate formulation. It can be washed after collection. Washing the seaweed will remove salt, sand and biological contaminants. Advantageously, seaweed is spun to get rid of excess water after washing. In embodiments, parts of the seaweed are used. For example, after harvesting, the upright portions may be cut off, leaving the rhizome/root-like structures. Chemical compounds may be extracted from the whole seaweed after collection and the compounds, alone or in a mixture, may be administered to an animal. Alternatively, the biomass remaining after extraction of the seaweed with a solvent can be administered to an animal. The seaweed, extract or the extracted biomass can be dried and administered as a powder, or the powder may be incorporated into a pellet as discussed above. In an embodiment, the seaweed is dehydrated or dried for storage. The seaweed may be dried in various ways including air drying, oven drying and freeze drying. Alternatively, the seaweed can be used fresh or wet in its whole form when available in this format.

Methods of extraction of chemical compounds from algae are well understood by the person skilled in the art. By way of example, solvent extraction may be employed. However, other techniques such as super-critical fluid extraction, in which the temperature and pressure of a fluid are raised above their critical point to give characteristics of both liquids and gases, may be used. The extraction may be assisted by exposing the material to high-pressure steam and/or a water-based solution containing water and/or other suitable solvents. The extraction process can also be effectively assisted by the application of a static and/or alternating physical field such as heat energy and/or a high-frequency alternating physical field, examples of which include but are not limited to microwave, radio-frequency or ultrasonic fields. By way of example, extraction may be assisted by the use of techniques such as the application of ultrasound waves with a frequency above 20 kHz to 100 kHz to break down the material. These waves cause the creation of bubbles and zones of high and low pressure. When bubbles collapse in the strong ultrasound field cavitation occurs. The implosive collapse, cavitation, near liquid-solid interfaces causes breakdown of particles, which means that mass transfer is increased, and bioactive compounds are released from biological matrix.

The extraction process typically comprises extraction with an organic solvent or water-based medium. The extraction process may involve, for example, an aqueous alkali-based leaching, but water or an organic solvent may be used. Mixtures of the treatment agents may be used if desired. The extraction may be at an elevated temperature. Typically, an extraction is conducted at a temperature from 30° C. to 80° C. Typically, the extraction time is from 24 hours to 72 hours.

The seaweed may be extracted with a polar solvent such as water, or a polar organic solvent such as an alcohol, in particular methanol, ethanol, propanol, butanol or hexanol, acetone, ethyl acetate, dimethylsulfoxide, dimethylformamide and tetrahydrofuran. In this case relatively more polar molecules will be extracted. The residue will contain relatively less polar compounds and the balance of the polar compounds not extracted under the extraction conditions used. Compounds that are active in inducing the effects described herein may be extracted but, equally, non-active or even compounds with deleterious effects may be extracted. In the former case it may be expected that the extract will have utility in the present invention, while in the latter it may be the residue that is effective. The skilled person will be able to ascertain with only routine experimentation which solvents are effective in producing an extract of greater effect than the seaweed itself.

The seaweed may be extracted with a non-polar solvent such as n-hexane, cyclohexane, benzene, toluene, chloroform, carbon tetrachloride or an ether such as diethyl ether. In this case relatively less polar molecules will be extracted. The residue will contain relatively more polar compounds and the balance of the non-polar compounds not extracted under the extraction conditions used. As for polar solvents, the skilled person will be able to ascertain with only routine experimentation which solvents are effective in producing an extract of greater effect than the seaweed itself.

Any reference to “seaweed”, “red seaweed”, “seaweed of Asparagopsis species” or similar in the context of this invention shall be taken to refer to the seaweed itself in any physical form as well as to extracts of the seaweed or the residue of the seaweed once extracted.

In an embodiment an extract of the seaweed with a polar solvent is used.

In an embodiment the extract of the seaweed is an extract with a polar solvent selected from the group consisting of water, an alcohol, acetone, ethyl acetate, dimethylsulfoxide, dimethylformamide and tetrahydrofuran.

In an embodiment the polar solvent is an alcohol, in particular methanol, ethanol, propanol, butanol or hexanol, and typically methanol.

In an embodiment the red seaweed, or an extract therefrom or the residue of the seaweed once extracted, is incorporated into an animal feed.

When used in combination with a feed material for birds, the feed material is s primarily made up of cereal grains (e.g. wheat, barley and sorghum) and oilseed meals (such as soya bean or canola meal) or animal by-product meals . . . . The feed is supplemented with the red seaweed of the invention. Thus, the animal, when feeding, ingests the red seaweed which can then act to increase production traits.

In an embodiment, the red seaweed, or an extract therefrom or the residue of the seaweed once extracted, is incorporated into fish feed which, in addition to the red seaweed, comprises one or more water soluble and/or dispersible nutritional ingredients. Typically, fish feed is in the form of a pellet or crumble which comprises one or more water soluble and/or dispersible nutritional ingredients and other ingredients. Typically the water soluble and/or dispersible nutritional ingredients are vegetable matter, e.g., flour, meal, starch or cracked grain produced from a crop vegetable such as wheat, alfalfa, corn, oats, potato, rice, and soybeans; cellulose in a form that may be obtained from wood pulp, grasses, plant leaves, and waste vegetable matter such as rice or soy bean hulls, or corn cobs; animal matter, e.g., fish and shellfish (e.g., shrimp or crab) meal, oil, protein or solubles and extracts, krill, meat meal, bone meal, feather meal, blood meal, or cracklings. Typically, a fish feed pellet further comprises ingredients such as binders, fillers, vitamins and minerals, amino acids, colourants, chelating agents and stabilisers. In addition, fish feed pellets can comprise antibiotics and other medicinal compounds.

In an embodiment fish feed pellets comprise red seaweed which has been comminuted.

In an embodiment fish feed pellets comprise red seaweed which has been dried and powdered. The powdered red seaweed can be sieved to in order to select seaweed particles of a particular size. In an embodiment, the powdered seaweed is reduced to a particle size of from 10 to 1000 microns. In an embodiment, the powdered seaweed is reduced to a particle size of from 100 to 500 microns. In an embodiment, the powdered seaweed is reduced to a particle size of about 400 microns. Seaweed powder can be incorporated into fish feed pellets.

In an embodiment the fish feed comprises red seaweed, an extract therefrom or residual biomass following an extraction process, in an amount from 0.1% w/w to 10% w/w. In an embodiment the fish feed comprises red seaweed in an amount of from 0.5% w/w to 5% w/w. In an embodiment the fish feed comprises whole red seaweed in an amount of from 1% w/w to 3% w/w. Typically the solvent extract has greater effect. In an embodiment, the whole seaweed is administered at 3% w/w while a 3% solvent extract is administered at 0.6% w/w. In an embodiment the fish feed comprises whole red seaweed in an amount of from 1% w/w to 3% w/w. In an embodiment the fish feed comprises an extract from the red seaweed. In an embodiment the extract is administered at 0.1% w/w to 1.5% w/w, preferably 0.5% w/w to 1.0% w/w.

It is preferable that fish be fed the red seaweed as a component of fish feed pellets, crumbles, or other fish feed forms, e.g., commercially available fish feed, or as an ingredient in a fish feed comprising other well-known ingredients included in commercial fish feed formulations so as to provide a nutritionally balanced complete fish feed, including, but not limited to: vegetable matter, e.g., flour, meal, starch or cracked grain produced from a crop vegetable such as wheat, alfalfa, corn, oats, potato, rice, and soybeans; cellulose in a form that may be obtained from wood pulp, grasses, plant leaves, and waste vegetable matter such as rice or soy bean hulls, or corn cobs; animal matter, e.g., fish and shellfish (e.g., shrimp or crab) meal, oil, protein or solubles and extracts, krill, meat meal, bone meal, feather meal, blood meal, or cracklings; vitamins, minerals, and amino acids; organic binders or adhesives; and chelating agents and preservatives.

In an embodiment feed pellets comprise components selected from a group consisting of proteins from plant meals such as meals derived from soy, corn and wheat, animal meals such as meat meal, blood meal and bone meal, and fishmeal; fish oil; vegetable oil (e.g. canola); binders; fillers; vitamins and minerals; and colourants.

In an embodiment the feed pellets comprise protein from fishmeal. In addition to its protein component, fishmeal also has a relatively high content of certain minerals, such as calcium and phosphorous, as well as certain vitamins, such as B-complex vitamins (e.g., choline, biotin and B12), and vitamins A and D. Industrial fishmeal usually also contains about 15% fish oil, which provides a source of important essential fatty acids.

In an embodiment the feed pellets comprise fish oil from the fishmeal and/or from other sources. Fish oil includes lipid-soluble vitamins (e.g., Vitamin A from fish liver oils) and certain preformed long chain polyunsaturated fatty acids (LC-PUFAs), such as arachidonic acid (ARA), eicosapentaenoic acid (EPA), and docosahexaenoic acid (DHA). Fish oil may be derived from wild caught fish or from other sources such as farmed fish or algal extracts.

The fillers and binders are used to bind the protein-rich ingredients together to improve stability in water. They are also useful in to improving the efficiency of the feed manufacturing process and to reduce feed wastage. Ingredients commonly used as binders in feed pellets include wheat gluten, sodium and calcium bentonites, lignosulfates, hemicellulose, carboxymethylcellulose, alginates, and guar gum. Binders such as bentonites, lignosulphonates, hemicellulose and carboxymethylcellulose reduce the frictional forces of the feed mixture as it passes through the pellet dies, thereby increasing the output and efficiency of the feed mill. Binders also increase the pellet hardness and reduce the formation of ‘fines’ during the pelleting process. Typical fillers include, for example, for example rice, soy, or wheat bran, rice, soy, or wheat flour, corn meal, rye, barley, sorghum, dextrose, sucrose, fructose, maltodextrin, starch or any combination thereof. Filler ingredients also often contain preservatives, such as, for example, ethoxyquin, which is often used as an anti-oxidant in fish feed.

Colorants are used in feed pellets for salmon to meet the consumer preference for red coloration in the flesh of the fish when it is consumed, but may also be used in other fish species. Carotenoid pigments such as astaxanthin or canthaxanthin are often used as colorants. In order to meet the consumer preference for red coloration, salmonid flesh should contain at least 5-20 mg pigment per kg flesh. To achieve these levels at least 40-60 mg of canthaxanthin or 40-150 mg astaxanthin has to be added per kg of feed.

Vitamins and minerals may be added to the feed pellets. The person skilled in the art will appreciate that the identity of and the amount of vitamins and minerals required will vary among species. Typically, one or more vitamins selected from the group consisting of vitamin A, vitamin C, vitamin D3, vitamin E, pantothenic acid, niacin, inositol, vitamin B2, vitamin B6, thiamine, folic acid, biotin, vitamin B12 will be added. Typically, minerals selected from the group consisting of zinc, manganese, iodine, copper and potassium. Minerals may be added as salts, for example the abovementioned minerals may be added in the form of zinc sulfate, manganese sulfate, ethylene diamine dihydroiodide, copper sulfate and potassium sorbate, respectively, as would be well understood by the person skilled in the art. Amino acid supplements may also be included. Most commonly, the amino acids added are the essential amino acids for fish. In an embodiment one or more amino acids selected from the group consisting of threonine, valine, leucine, isoleucine, methionine, tryptophan, lysine, histidine, arginine and phenylalanine is added.

A typical feed formulation for fish in the grow out stage would generally include a protein source such as fish meal, defatted soybean meal, or poultry meal. It will also contain a carbohydrate source, with wheat meal, corn-starch, rice bran being popular options, and a lipid source including fish and vegetable oil. The feed will also contain a vitamin and mineral mix (vitamin A, C, D3, E, K3, B1, B3, B6, B5, B12, folic acid, inositol, biotin, copper sulfate, magnesium oxide, manganese sulfate, potassium iodide, iron sulphate, zinc oxide, dextrose and the antioxidant oxicap E2), mould inhibitor and amino acids supplements.

In embodiments Asparagopsis altered the community composition of the microbes in the fish intestine. The use of seaweed of Asparagopsis species as an additive in fish feed provides an alternative way to manage the intestinal microbial flora, or the gut microbiome. Asparagopsis species lead to an increase in Firmicutes bacteria and a decrease in Proteobacteria. When included orally as a raw ingredient at 3% w/w of the feed, the Arcobacter sp. (Proteobacteria, potential fish and human gastrointestinal pathogen) was 15× less abundant in fish fed Asparagopsis species than in the fish fed the control diet. Bacteria of the family Lachnospiraceae (Firmicutes) were about 4 times more abundant in the Asparagopsis species fed fish than in the fish fed the control diet. The dominant Operational Taxonomic Unit (OTU) in the fish fed the control diet in the Asparagopsis extract trial (described in Example 5) was a bacteria with an unidentified genus of the family Erysipelotrichaceae, which represented 44% of the hindgut OTUs. Erysipelotrichaceae was reduced in all treatments with a maximum of 41% abundance in the 3% whole seaweed fed fish. Lachnospiraceae was relatively low in abundance in the control fed fish in both the first and second rabbitfish trial. Lachnospiraceae was abundant in the seaweed, whole, extract or residue fed fish; up to 4 times for 3% whole A. taxiformis fed fish in the first trial, and up to 3 times for the 6% extract fed fish in the second trial. Compared to the control diet fish, the Asparagopsis extract and residue fed fish led to almost doubling the abundance of Desulfovibrionaceae.

The alpha diversity of the microbial community in the hindgut of the fish fed Asparagopsis whole was enhanced by 15% compared to the fish fed the control diet. The relative abundance of Tenacibaculum sp. (potential fish pathogen) was reduced by 93% in the Asparagopsis extract trial for the fish fed the seaweed whole (6%), extract (1.5%) and residue (3%) compared to the control fish. Additionally, Clostridium sp. was reduced by 70% in the fish fed the whole (3%) diet compared to those fed the control diet. In Atlantic salmon (described in Example 6), the fish fed Asparagopsis (whole and extract) had higher alpha diversity (by up to 70%) compared to the fish fed the control diet. The fish fed the whole and extract seaweed at 3% had no Escherichia/Shigella sp. compared to the control fish which has a relative abundance of 4.4% for this bacteria. The salmon fed the seaweed diet whole (3%) and extract (6%) had up to 123% increase in relative abundance of Shewanella sp. in their hindgut compared to the control fish.

The red seaweed, or an extract therefrom or the residue of the seaweed once extracted, may be formulated as a veterinary formulation. Such a formulation may be administered by any route suitable for the animal. By way of example, as appropriate, it may particularly be administered orally, intravenously, intramuscularly, cutaneously, subcutaneously, transmucosally (e.g. sublingually or buccally), rectally, transdermally, nasally, pulmonarily (e.g. tracheally or bronchially), or topically.

The red seaweed, or an extract therefrom or the residue of the seaweed once extracted, can be administered as a veterinary formulation in admixture with an adjuvant, diluent or carrier, which may be selected with due regard to the intended route of administration and standard veterinary practice. Such carriers may be chemically inert and should have no detrimental side effects or toxicity under the conditions of use.

Otherwise, the preparation of suitable formulations may be achieved routinely by the skilled person using routine techniques and/or in accordance with standard and/or accepted veterinary or pharmaceutical practice.

In an embodiment, the red seaweed, an extract therefrom or the residue of the seaweed following an extraction process, is administered in conjunction with another additive to boost production traits.

The person skilled in the art will appreciate that algae, extracts or residues and/or commercial additives may be administered sequentially, simultaneously or concomitantly This may involve administration of separate formulations, be they animal feeds or veterinary formulations, with one containing the red seaweed, and another containing the other active ingredient. These may be provided together a kit of parts with instructions for use. Alternatively, it might involve administration of a single animal feed or veterinary formulation containing the red seaweed and the other component.

### EXAMPLES

Hereinafter, embodiments of the invention are illustrated in more detail with reference to the following examples. However, the present disclosure is not limited thereto. Furthermore, what is not described in this disclosure may be sufficiently understood by those who have knowledge in this field and will not be illustrated herein.

### Example 1 Collection of Seaweed and Preparation of Fish Feed

Eleven species of seaweed (marine macroalgae) from three taxonomic groups (green, brown and red) were evaluated. All treatments were delivered as a 3% w/w dietary inclusion in the fish feed. The origin, treatment and processing of the seaweeds used in this study is summarised in Table 1. Two positive controls were used in this study namely Hilyses®, a hydrolysed yeast culture derived from the sugarcane fermentation process, and sodium alginate, the anionic polysaccharide extracted from brown seaweeds. In addition, the cyanobacteria spirulina (Swisse high strength organic Spirulina) and the microalgae Haematococcus pluvialis were also included as positive control as often used in fish studies. All seaweeds were rinsed with salt water to remove sand and biological contaminants. They were then spun to get rid of excess water and frozen at −80° C. before being processed in a freeze dryer (Thermo Savant model MODULYOD-230) for at least 3 days at approximately −44° C. and 206 mbar. Once dried, all seaweed species were vacuum sealed in individual bags with desiccant and stored at −20° C. until needed. The control diet was produced based on the commercial pellet Native (Ridley Aquafeeds Ltd). The Native pellets were first powdered and then warm water was slowly added in a blender (Hobart A120) for approximately 10 min to produce a stiff dough. The dough was extruded through a 4 mm die on trays which were then oven dried overnight at 50° C. Once dried the feed was crumbled and packaged in airtight bags, subsequently stored at 4° C. for the duration of the trial. The experimental diets were made in the same manner but received the powdered and 300 μm sieved ingredients (Table 1) prior to adding the water during the blending step. Table 1 contains a list of ingredients trialled.

| Species | Morphology | Processing |
|---|---|---|
| Sarconema sp. | Soft fleshy, branching | Freeze dried |
| Gracilaria sp. | Soft fleshy, branching | Freeze dried |
| Kappaphycus sp. | Rigid, branching | Sun dried |
| Asparagopsis sp. | Soft, fleshy | Freeze dried |
| Sargassum sp. | Fleshy, turgid | Freeze dried |
| Dictyota sp. | Fine, dichotomously | Freeze dried |
| | branching | |
| Lobophora sp. | Tough, leathery, thallus | Freeze dried |
| Halimeda sp. | Hard, calcifying | Freeze dried |
| Caulerpa sp. | Soft, fleshy | Freeze dried |
| Ulva sp. | Single cell thick blade | Freeze dried |
| Cyanobacteria | | |
| Spirulina sp. | Marine filamentous blue- | |
| | green algae | |
| Microalgae | | |
| Haematococcus | Unicellular, spherical cysts | Refractance |
| pluvialis | (cracked biomass) | window dried |

### Example 2 Fish Collection and Feeding

The juvenile rabbitfish, Siganus fuscescens, were captured using a drag net (15 m long by 2.1 m deep with a 2.5 cm mesh size). All fish were collected at Moffat Beach, Queensland Australia (26° 47′21.7″S 153° 08′36.0″E) on rocky reefs off the beach and transferred to the Bribie Island Research Centre (BIRC) in an oxygenated 500 L fish carrier. Once at BIRC, they received a hydrogen peroxide bath (200 mg/l for 30 min) to rid the fish of potential external pathogens and parasites. After treatment, the fish were transferred in three 1000 L fibreglass tanks where they were acclimatised and fed the control diet for two weeks. For the screening trial, 144 fish in total made up of three groups of 48 fish (small, medium and large). The initial fish weight for each group was 85.83±7.85 g, 112.60±8.17 g and 150.59±14.59 g for the small, medium and large fish groups respectively. The fish were randomly allocated into 48 plastic tanks (55 l) at a rate of 3 fish per tank, with one fish from each group. One replicate tank per treatment was stocked each day over three days. The diets were hand fed at 3% w/w body weight twice a day (10:00 a.m. and 3:00 p.m.). During the trials, the water temperature was maintained at 27° C. and pH in a range of 7.9 to 8.1. The system was operated as flow through using seawater pumped approximately 300 m off the beach adjacent to the station. The seawater is then pumped through a series of 16 spin disk filters (40 μm) and 10 multimedia filters (˜10-15 μm), after which it receives ozone treatment from two 100 gO₃/h generator units (WEDECO OCS-GSO30). The ozone treated seawater is then pumped via ultra violet filters, providing 80 mJ/cm², to two 4×2.2 m granular activated carbon vessels for a contact time of >9 mins to remove unwanted by-products from the ozone treatment. Finally, the seawater is pumped to a header tank, which fed directly into a pipe system delivering treated seawater to this experiment. The system was in a temperature and light controlled room kept at 24-26° C. and on a 24:0 L:D light regime.

### Example 3 Production of Juvenile Fish

The juvenile rabbitfish Siganus fuscescens used in this experiment were raised from eggs from wild captured broodstock. All broodstock fish were collected at Moffat Beach, Queensland Australia (26° 47′21.7″S 153° 08′36.0″E) on rocky reefs off the beach. They were then transferred to the Bribie Island Research Centre in an oxygenated 500 L fish carrier. Once at Bribie Island Research Centre, they received a hydrogen peroxide bath (200 mg/l for 30 min) to rid the fish of potential external pathogens and parasites. After treatment, the fish were transferred to a 1000 L fibreglass tank outside to be exposed to natural light and moon cycles. The fish were fed at 3% body weight per day of the Native range (Ridley Aquafeeds Ltd) over 6 feeds per day for a period of 8 month before the first natural spawn.

Eggs from a natural spawn were collected in a 300 μm mesh egg collector, incubated and hatched in a gently aerated 1000 L conical tank. The newly hatched larvae were then concentrated using a 300 μm mesh internal standpipe and transferred to three matured 3000 L parabolic mesocosm tanks. One month prior to the spawn, the mesocosm tanks were incubated with microalgae (Nannochlorpsis occulata and Tetraselmis chui) and fertilizer (Microalgae food, Manutec). The small strain rotifer Brachionus rotoduntiformis were dosed in the mesocosm tanks at 1 day post hatch (dph) at a density of 5 rotifers/ml. The fish larvae started to feed soon after their mouth open at 2 dph. The rest of the larval rearing used standard marine fish larval rearing protocols.

At 30 dph, the fish were graded and transferred to an indoor light and temperature controlled room in 1000 L tanks to acclimate to the change of conditions before being stocked in 50 L experimental tanks. Two weeks before the start of the productivity trial, the medium size fish from the medium grade were stocked in the experimental tanks and fed the control diet, one replicate tank per day over four days. From that point until completion of the experiment, the water temperature was maintained at 27° C. and pH in a range of 7.9 to 8.1. The system was operated as flow through using seawater pumped approximately 300 m off the beach adjacent to the station. The seawater was then pumped through a series of 16 spin disk filters (40 μm) and 10 multimedia filters (˜10-15 μm), after which it receives ozone treatment from two 100 gO3/h generator units (WEDECO OCS-GSO30). The ozone treated seawater was then pumped via ultra violet filters, providing 80 mJ/cm2, to two 4×2.2 m granular activated carbon vessels for a contact time of >9 mins to remove unwanted by-products from the ozone treatment. Finally, the seawater was pumped to a header tank, which fed directly into a pipe system delivering treated seawater to this experiment. The system was in a temperature and light controlled room kept at 24-26° C. and on a 12 L:12D light regime.

### Example 4 Microbiome Analysis

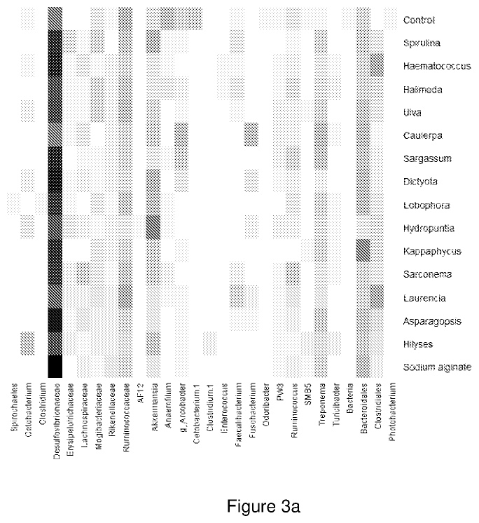

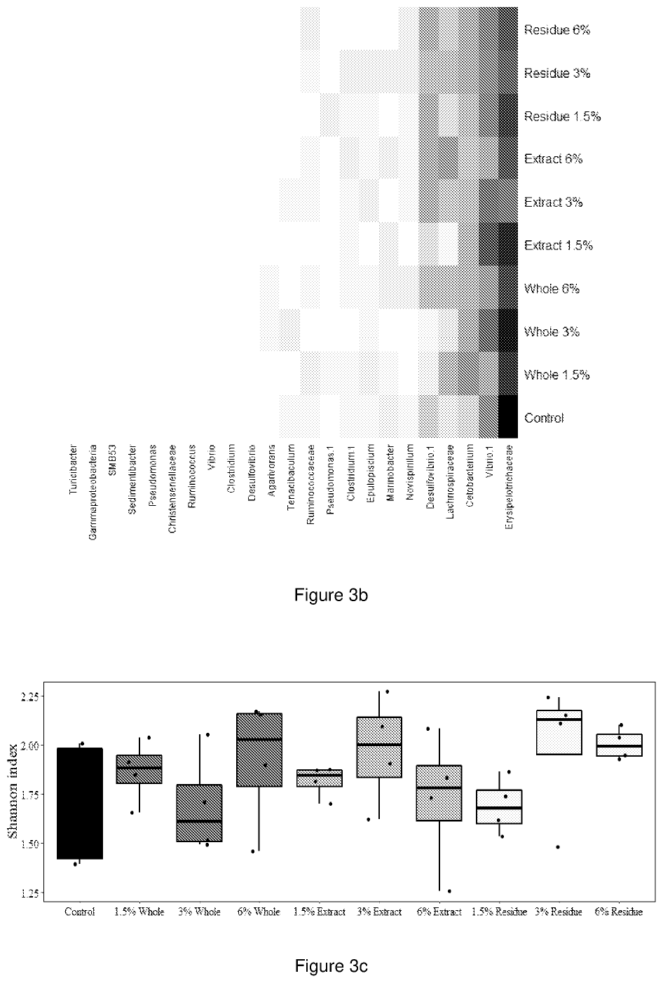

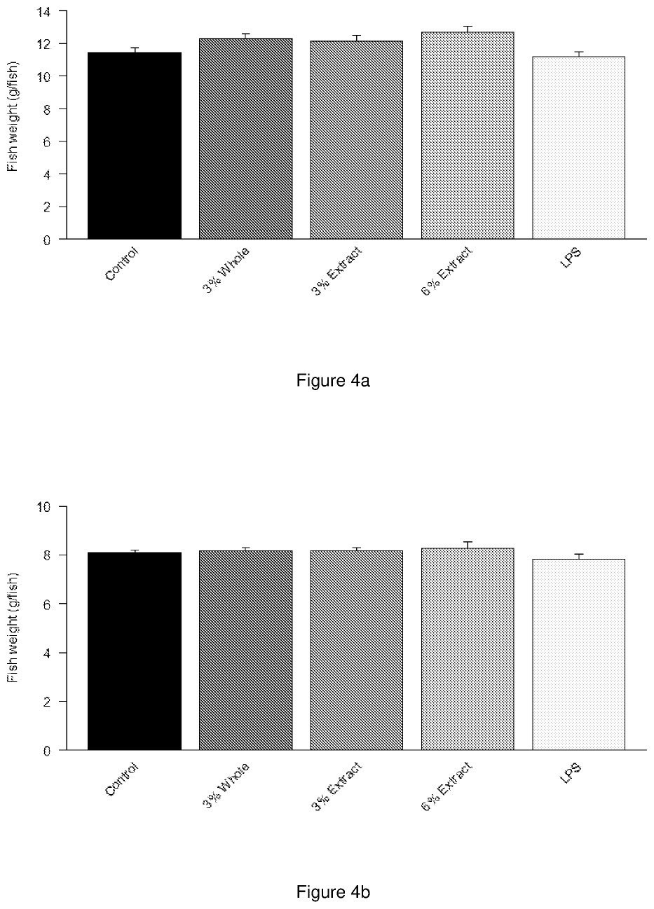

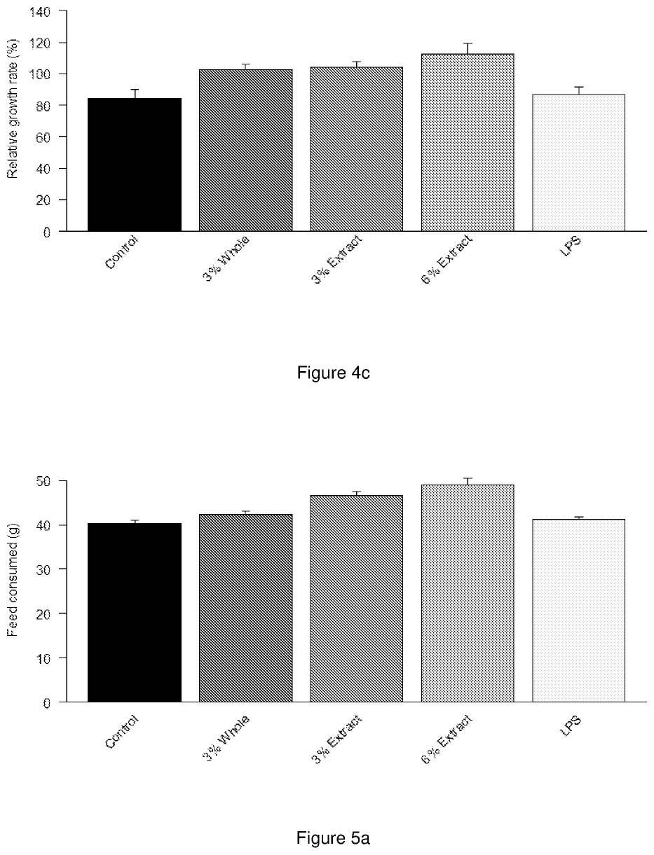

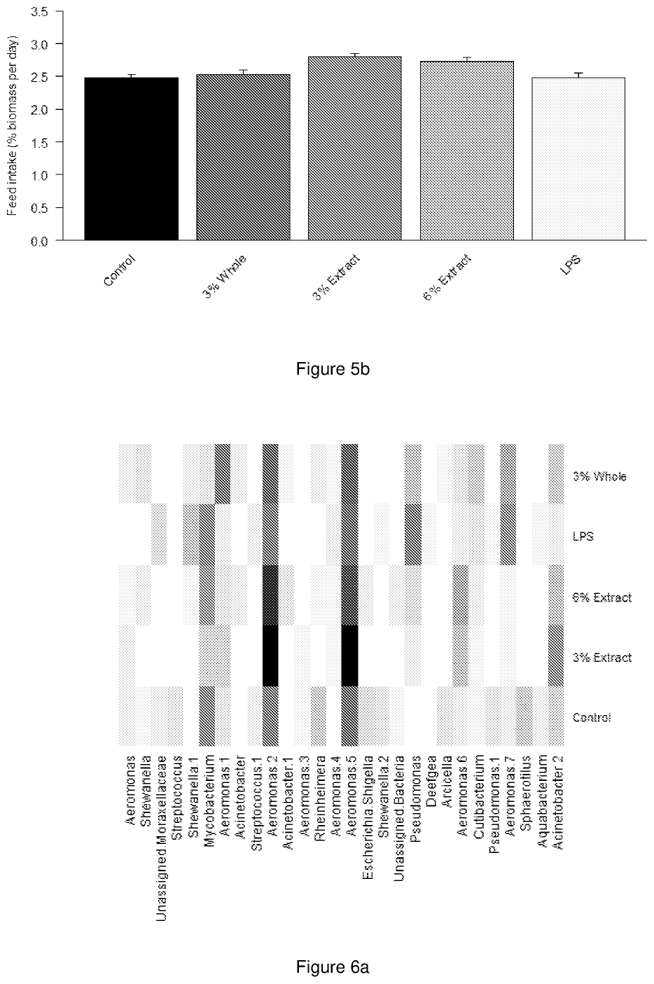

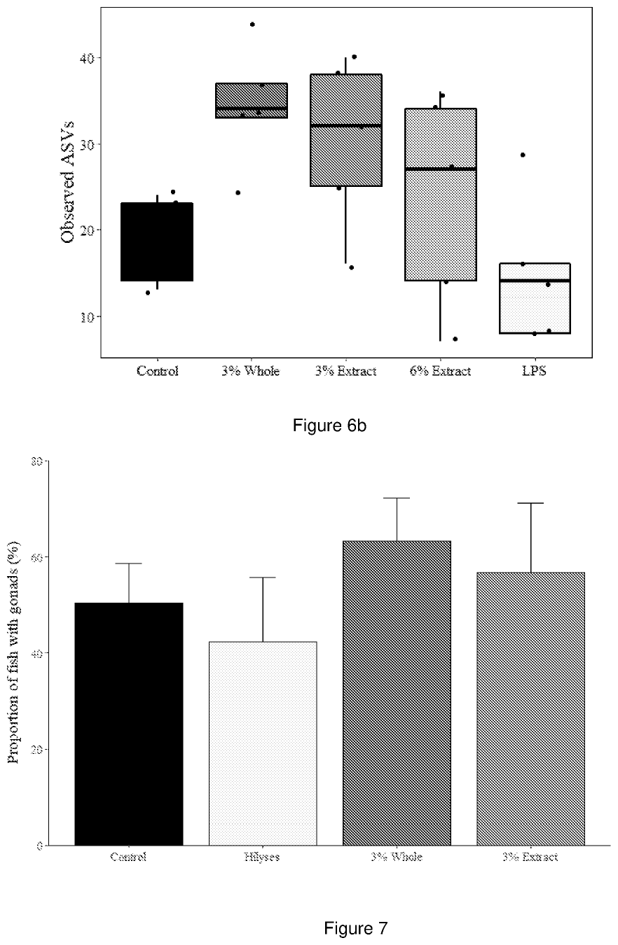

After a 24 h fasting period the fish from one replicate tank per treatment was harvested each day over a three days period. The whole gut from each fish was aseptically removed and placed in a 50 ml centrifuge tube before being frozen and stored at −80° C. until further processing could occur. The gut samples were thawed and 0.25 g, approximately 0.5 cm, of hindgut taken starting 1 cm away from the anus were taken from each selected fish and placed directly in powerbeads tubes from the PowerSoil® DNA isolation kit (Mo Bio, San Diego, Calif., USA). The DNA was extracted following the manufacturer's instructions and thereafter stored at −20° C. The microbial diversity profiling of the variable region V3-V4 using the forward primer 341F (CCTAYGGGRBGCASCAG) and the reverse primer 806R (GGACTACNNGGGTATCTAAT) of the 16S rRNA gene was performed by the Australian Genome Research Facility. The sequencing was performed on a MiSeq platform (2×300 bp) and the resulting reads were analysed with Illumina bcl2fastq pipeline version 2.20.0.422bcl2fastq pipeline version 2.20.0.422 (2×300 bp miseq platform). Demultiplexed paired-end reads were assembled by aligning the forward and reverse reads using Quantitative Insights into Microbial Ecology (QIIME2 v2018.8). To ensure that comparisons were made from sequences assigned in the same hypervariable region (V4) of the comparison studies (below), the raw data from the current study was trimmed using the cutadapt package (64), using the 515F (5′-GTGCCAGCMGCCGCGGTAA-3′) and 806R (5′-GGACTACHVGGGTWTCTAAT-3′) primers. Trimmed sequences were processed and denoised using the DADA2 package and QIIME2 (v2018.8) software, with ASVs tables constructed and aligned against the Silva 16S rRNA 99% reference database (release v132). We found specific activity of Asparagopsis in changing the intestinal microbial flora. In comparison with the control treatment, the microbial community composition of Asparagopsis was altered. For example, bacteria of the genus Arcobacter sp. was 15× less abundant than that of the control fish, while Romboutsia sp. and was 8× more abundant than of the control fish (FIG. 3). All seaweed treatments and positive controls were affected in similar ways compared to the negative control with Asparagopsis sp. not being noticeably different from the other treatments.

### Example 5 Effect of Dietary Asparagopsis taxiformis Supplementation on the Growth and Stress Resistance of Siganus Fuscescens

The purpose of this experiment was to evaluate the dose response effects of whole Asparagopsis seaweed and a methanol extract on the growth rate and stress resistance of fish using the mottled rabbitfish as model. The Asparagopsis sample used was from the collection batch referred to in Example 1. Once powdered, the seaweed was added at 1.5, 3 or 6% w/w into commercial aquafeed or extracted with methanol. For the extract, the methanol was evaporated and the yield from the whole biomass was 20% w/w. The dried extract was then resuspended in hexane and water. This suspension was added to the commercial aquafeed at the respective whole seaweed dose (1.5, 3 and 6%). The residual biomass from the extraction (“residue”) was dried. The residue comprised 80% w/w of the whole biomass. This was then added to the commercial aquafeed at the respective whole seaweed dose (1.5, 3 and 6%). The results of percentage change observed in the treatment compared to the control diet in key measurements by the end of the 4 weeks trial are set out in Table 2

| | Effective | | |
|---|---|---|---|
| | % feed | Fish | |
| Treatment | displacement | weight | Survival |
| 1.5% whole | 1.5% | +7% | +119% |
| 3% whole | 3% | +3% | +85% |
| 6% whole | 6% | +4% | +105% |
| 1.5% Extract | 0.3% | +11% | +130% |
| 3% Extract | 0.6% | +9% | +126% |
| 6% Extract | 1.2% | +17% | +157% |
| 1.5% Residue | 0.3% | +8% | +106% |
| 3% Residue | 0.3% | +2% | +110% |
| 6% Residue | 0.3% | +10% | +127% |

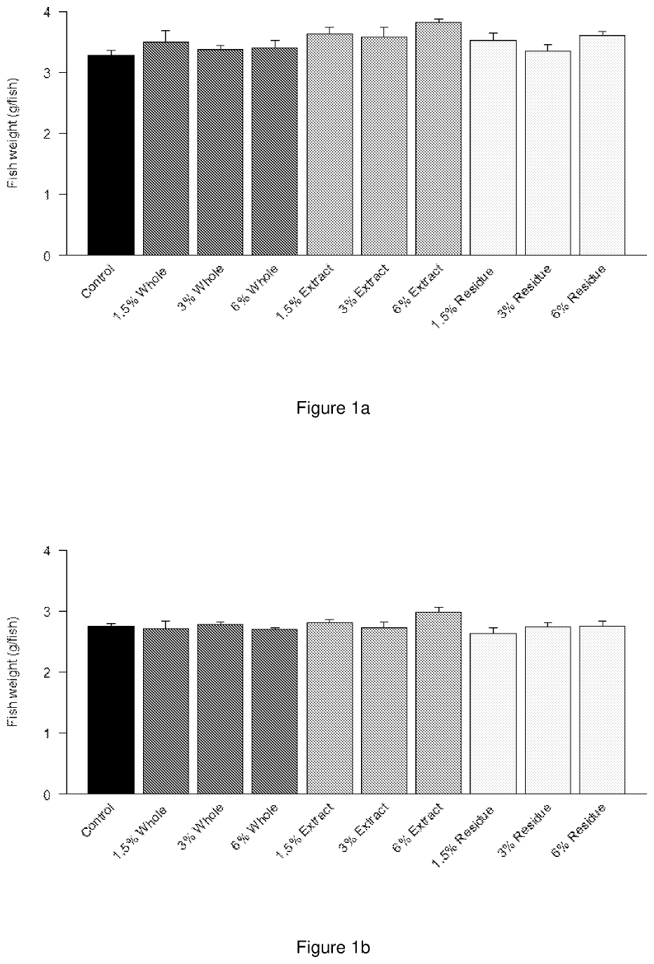

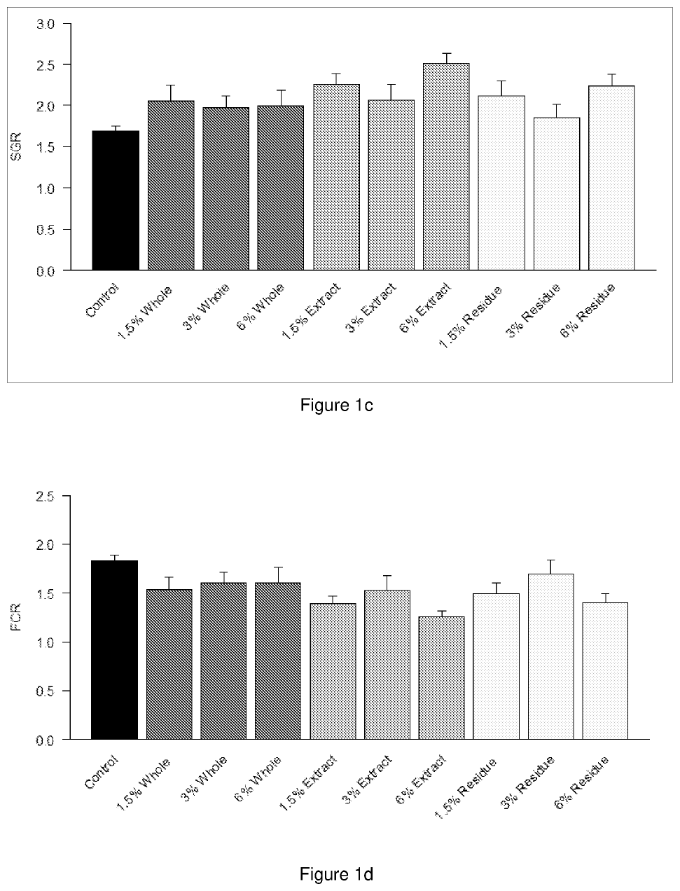

Most treatments had positive effects on growth. However, the best performing group was the natural product extract at the 6%-whole equivalent dose (FIG. 1, “6% Extract”; average 3.8 g/fish). These fish doubled their weight during the 4-week experiment. The control, un-supplemented diet was the worst performing treatment (average 3.2 g/fish).

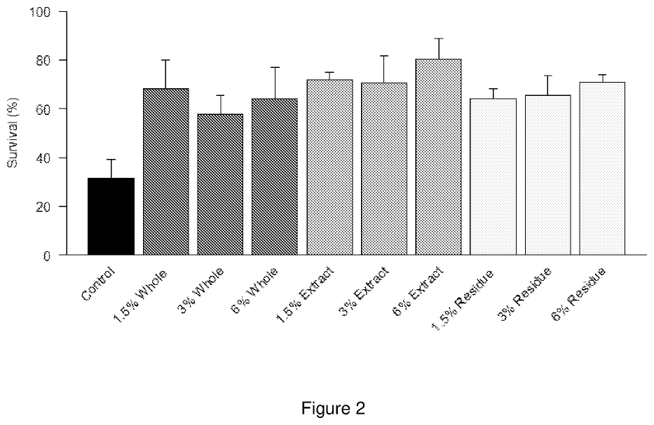

The survival/stress challenge was improved in all treatments compared to the control (FIG. 2, 31.2%). The extracts were the best performing treatments, with again the 6% extract as the best performing treatment (“6% Extract”; 80.3%).

### Example 6 Effect of Dietary Asparagopsis taxiformis Supplementation on the Growth and Feeding Rates of Salmo Salar

A production trial similar to Example 4 was conducted for Atlantic salmon (Salmo salar). Asparagopsis taxiformis was collected from Moffat Beach, Queensland Australia (26° 47′21.7″S 153° 08′36.0″E). The seaweed was cleaned using seawater to remove sand and epiphytes before being spun to remove excess salt water. Following this the seaweed was frozen at −80° C. before being processed in a freeze dryer (Thermo Savant model MODULYOD-230) for at least 3 days at approximately −44° C. and 206 mbar. Once dried, the seaweed was powdered and kept in a vacuumed sealed bag in the −80° C. until future use. The control diet was produced based on the commercial Nutra Supreme-RC (Skretting Ltd). The Skretting pellets were first powdered and then warm water was slowly added in a blender (Hobart A120) for approximately 10 min to produce a stiff dough. The dough was extruded through a 2 mm die on trays which were then dried in a fan driver food dehydrator (Sunbeam) at room temperature for 12 h. Once dried the feed was packaged in airtight bags and subsequently stored at 4° C. for the duration of the trial. The experimental diets were made in the same manner but received the powdered and 300 μm sieved seaweed or the seaweed extract prior to adding the water during the blending step. The seaweed extract was made using 150 g of freeze dried A. taxiformis, which was extracted 4 times over 12 h in 500 ml of methanol. The 1 l extract rich methanol was then slowly evaporated using a rotary evaporator with the extract sitting in a 30° C. bain-marie. Once the methanol fully evaporated the extract was resuspended in 400 ml of deionised water and 100 ml of hexane. The extract for the two extract treatments (3% and 6%) was added at the equivalent proportion of whole seaweed as per Example 1. A positive control lipopolysaccharide (LPS from Escherichia coli, purchased from Sigma) was added at 0.01% w/w into the feed.

The salmon fry, Salmo salar (5 g), were shipped from a hatchery in Tasmania to the Bribie Island Research Centre (BIRC). Once at BIRC, they were spread in between two 1000 L fiberglass conical tanks where they remain for and acclimation period of 6 days. The fish were then randomly allocated into 50 plastic tanks (55 l) at a rate of 18 fish per tank. The diets were hand fed to satiation twice a day (10:00 a.m. and 3:00 p.m.). During the trials, the water temperature was maintained at 15° C. by a heat pump (Oasis C16) and pH in a range of 7.0 to 7.8. The system was operated as a recirculating aquaculture system using dechlorinated town water and comprised of two Waterco C50 bag filters in parallel (50 um bags) followed by a Micron S602e sand-filter. The system was in a temperature and light controlled room kept at 18° C. and on a 12:12 L:D (08:00-20:00) light regime with a 30 min ramp up/down period.

After two weeks on the treatment and control diets the fish were fasted for 24 h prior to sampling. All fish in the replicate tanks were weighed and returned to their tanks for another two weeks on their treatment diets. This was again followed by a 24 h fasting period after which the final samples and weights were taken.

The fish feeding rate was calculated based on the number of spoons of feed given to the fish (1 spoon=0.8 g). The fish were fed to satiation (demand feeding) and thus it was assumed that the lack of feeding activity (rapid movement to chase pellets) meant that the fish were no longer hungry and no longer required feeding.

Percentage change observed in the treatment compared to the control diet in relative weight gain (% of starting weight) after 2 and 4 weeks feeding trial. The results are set out in Table 3

| | Effective | Fish | Fish |
|---|---|---|---|
| | % feed | weight | weight |
| Treatment | displacement | (4 weeks) | (2 weeks) |
| 3% whole | 3% | +22.4% | +13.2% |
| 3% Extract | 0.6% | +18.6% | +20.6% |
| 6% Extract | 1.2% | +29.6% | +24.8% |
| LPS | 0.01% | +3.6% | +0.9% |

Percentage change observed in the treatment compared to the control diet in relative feed intake (feed given as % of fish biomass) after 2 and 4 weeks feeding trial. The results are set out in Table 4

| | | % change | % change |
|---|---|---|---|
| | Effective | relative to | relative to |
| | % feed | control | control |
| Treatment | displacement | (2 weeks) | (4 weeks) |
| Whole 3% | 3% | +5.4% | +4.0% |
| Extract 3% | 0.6% | +18.9% | +8.8% |
| Extract 6% | 1.2% | +19.8% | +11.2% |
| LPS | 0.01% | +7.1% | −14.2% |

### Example 7 Chemical Analysis of the Methanolic Extract of Asparagopsis

The methanolic extract was created as per Example 5. The key natural products were analysed using Gas Chromatography Mass Spectrometry. The method for analysis was as follows: In order to make the seaweed extract treatments used in feed, a sample of freeze-dried Asparagopsis taxiformis was extracted in methanol in a round-bottom flask and the methanol driven-off under nitrogen stream. The residue was subsequently extracted in methanol and the process repeated for a total of 4 extractions. The extracts were combined, filtered and then subjected to rotary evaporation under vacuum until all methanol was driven-off. The approximate recovery of extract was 20% by mass of the original freeze-dried sample of Asparagopsis taxiformis. The extract was reconstituted in methanol with ethyl benzoate as internal standard, filtered and vialled for Gas Chromatography-Mass Spectrometry analysis. A portion of the original freeze-dried sample of Asparagopsis taxiformis (whole seaweed treatment) was taken for direct analysis by Gas Chromatography-Mass Spectrometry. For the whole seaweed treatment, the sample was directly extracted in methanol with ethyl benzoate as an internal standard, filtered and vialled for Gas Chromatography-Mass Spectrometry analysis.

Gas Chromatography-Mass Spectrometry was performed on a Perkin Elmer Clarus SQ8S fitted with a DB-5 column (Perkin Elmer Elite-5MS, 30.0 m×0.25 mm, 025 μm). Injections (1.0 μL) were introduced with a 50:1 split ratio with a sample rate of 1.56250 pts/sec. The GC was held at 40.0° C. for 1 min, ramped at 20.0° C. min-1 to 250.0° C. and held for 0 min followed by a 0.5 min equilibration time prior to the next injection. Helium was used as the carrier gas with a flow rate of 1 mL/1 min. Mass spectrometry was performed on a Perkin Elmer Clarus 580 across a weight range of 50-340 m/z. Analysis occurred from 3.0-12.0 min with a scan rate of 0.3 s. Compounds were identified by referencing mass spectral chromatographs to the NIST library. G.C. confidence intervals were then averaged across samples as well as within samples using different areas of the peak and subtraction of background ion profiles. Relative quantitation was achieved by comparison of peak area ratios (as determined using supplied TurboMass software) of compound to internal standard (equivalent to parts per million or compound (mg)/solvent (L)) which were then evaluated to give compound (g)/algae material (g).

In the table following, the percentages represent the relative abundance of the top 10 compounds detected in the whole Asparagopsis seaweed and in the extract, summing to 100%. Note that compound names denoted with (*) represent unique compounds not found in top 10 of both samples (unidentified compounds are indicated as compounds 1-6 with most likely halogenation). Note that there are up to 4 unique compounds in the list of the top 10 most abundant halogenated compounds in whole seaweed, compared to up to 3 unique compounds in the list of the top 10 most abundant halogenated compounds in the solvent extract. Key compounds detected in Asparagopsis and its methanolic extract are set out in Table 5

| | Whole Asparagopsis | | Solvent extract | |
|---|---|---|---|---|
| # | Compound | % | Compound | % |
| 1 | Bromoform | 68% | Tribrominated | 32% |
| | (tribromomethane) | | compound 1 | |
| 2 | Tribrominated | 18% | Dibromoacetic acid | 27% |
| | compound 1 | | methyl ester * | |
| 3 | Dibromoiodomethane | 4% | Bromoform | 9% |
| | | | (tribromomethane) | |
| 4 | Dibromoacetic acid * | 2% | Dibrominated | 6% |
| | | | compound 6 | |
| 5 | Halogenated | 2% | Tribromoacetic acid | 6% |
| | compound 2 | | methyl ester * | |
| 6 | Dibromochloromethane* | 2% | Dibromoiodomethane | 5% |
| 7 | Dibrominated & | 1% | Dibromobutenedioic | 4% |
| | chlorinated compound | | acid * | |
| | 3 * | | | |
| 8 | Dibrominated compound | 1% | Halogenated | 4% |
| | 4 | | compound 2 | |
| 9 | Tetrabromomethane * | 1% | Dibrominated | 4% |
| | | | compound 5 | |
| 10 | Dibrominated compound | 1% | Bromoacetic acid | 3% |
| | 5 | | | |
| | % total | 100% | % total | 100% |

### Example 8 Effect of Dietary Asparagopsis taxiformis Supplementation on the Fertility of the Rabbitfish Siganus Fuscescens

Asparagopsis taxiformis was collected from Moffat Beach, Queensland Australia (26° 47′21.7″S 153° 08′36.0″E). The seaweed was cleaned using seawater to remove sand and epiphytes before being spun to remove excess salt water. Following this the seaweed was frozen at −80° C. before being processed in a freeze dryer (Thermo Savant model MODULYOD-230) for at least 3 days at approximately −44° C. and 206 mbar. Once dried, the seaweed was powdered and kept in a vacuumed sealed bag in the −80° C. until future use. The control diet was produced based on the commercial Native (Ridley Aquafeeds Ltd). The Ridley pellets were first powdered and then warm water was slowly added in a blender (Hobart A120) for approximately 10 min to produce a stiff dough. The dough was extruded through a 2 mm die on trays which were then dried in a fan driver food dehydrator (Sunbeam) at room temperature for 12 h. Once dried the feed was packaged in airtight bags and subsequently stored at 4° C. for the duration of the trial. The experimental diets were made in the same manner but received the powdered and 300 μm sieved seaweed or the seaweed extract prior to adding the water during the blending step. The seaweed extract was made using 150 g of freeze dried A. taxiformis, which was extracted 4 times over 12 h in 500 ml of methanol. The 1l extract rich methanol was then slowly evaporated using a rotary evaporator with the extract sitting in a 30° C. bain-marie. Once the methanol fully evaporated the extract was resuspended in 400 ml of deionised water and 100 ml of hexane. The extract for the extract treatment (3%) was added at the equivalent proportion of whole seaweed as per Example 1. A positive control Hilyses® (β-glucan rich extract from Saccharomyces cerevisiae) was added at 3% w/w into the feed.

The adult rabbitfish were grown from eggs from a spawn the Bribie Island Research Centre which occurred on the 15th of January 2019. The larvae were reared using standard marine fish larval rearing protocol and the fish were grown until, 9 month later, they started to spawn (November 2019). This was a sign that at least a few fish were sexually mature so the fish (9 months old) were stocked in the experimental tanks. Twelve×1000 L tanks received 10 fish with each group comprising at least two large individuals (one ˜100 g and one ˜120 g) which were assumed to be females. The average fish weight per tank was reasonably consistent, ranging from (mean±SE) 73.30±5.74 g to 81.60±8.45, so that tank biomass was not significantly different between treatments (n=3 replicate tanks). The tanks were operated as flow-through seawater (filtration as described above) without temperature regulation of the water. Tank water temperature ranged from 26.1° C. to 29.2° C.) with an average of 27.7° C. over the course of the feeding trial, and salinity remained around 35 ppt. The fish received the experimental diets twice a day (10:00 and 14:00) and were fed to satiation for three months until sampling.

After 3 months of feeding on the experimental diets, the fish were sampled over two days and euthanized using 10 ppt Aqui-S®. The fish were then dissected and both females and male with distinct sexual organs were recorded to calculate the proportion of sexually mature individuals solely based on the presence or absence of distinct sexual organs. The fish fed the whole Asparagopsis diet had a 13% higher proportion of fish with mature gonads compared to the control fish (FIG. 7)

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

### REFERENCES

References cited herein are incorporated herein by this reference and are as follows:
Angell, A R, Mata L, de Nys R, Paul, N A The protein content of Seaweed: a universal nitrogen-to-protein conversion factor of five J Appl Physiol (2016) 28511-524
Paul N A, de Nys R, Steinberg P D (2006) Chemical defence against bacteria in the red alga Asparagopsis armata: linking structure with function. Marine Ecology Progress Series, 306, 87-101.
Verges A, Paul N A, Steinberg P D (2008) Sex and life-history stage alter herbivore responses to a chemically defended red alga. Ecology 89, 1334-1343.
Angell, A. R., Angell, S. F., de Nys, R., & Paul, N. A. (2016). Seaweed as a protein source for mono-gastric livestock. Trends in food science & technology, 54, 74-84.

1.-23. (canceled)

24. A method for increasing a measure of a production trait in a non-ruminant animal, comprising the step of administering to said animal an extract of a red seaweed of Asparagopsis species or the residue of the red seaweed following an extraction process.

25. The method of claim 24, wherein the red seaweed is Asparagopsis taxiformis.

26. The method of claim 24, wherein an extract of the red seaweed with a solvent is administered.

27. The method of claim 26, wherein the solvent is a polar solvent.

28. The method of claim 27, wherein the polar solvent is selected from the group consisting of water, an alcohol, acetone, ethyl acetate, dimethylsulfoxide, dimethylformamide and tetrahydrofuran.

29. The method of claim 28, wherein the polar solvent is an alcohol, or wherein the polar solvent is methanol.

30. The method claim 24, wherein a residue of the red seaweed following extraction with a solvent is administered.

31. The method claim 24, wherein administration of the red seaweed is by feeding the animal an animal feed containing the red seaweed.

32. The method of claim 31, wherein the animal feed further comprises components selected from a group consisting of proteins, fish oil, binders, fillers, vitamins and minerals, amino acid supplements, colourants, chelating agents, and preservatives.

33. The method of claim 24, wherein the animal is a bird.

34. The method of claim 33, wherein the bird is selected from the group consisting of chickens, quails, turkeys, emus, fowls such as peafowl and guinea fowl, swans, turkey, geese, ducks, ostriches, pheasants, partridges, and pigeons.

35. The method of claim 24, wherein the animal is one of the bony fishes, particularly one of the teleosts.

36. The method of claim 35, wherein the animal is a fresh, brackish, or salt water fish species including, but not limited to: barramundi, catfish, carp, trout, salmon, tuna, cobia, char, whitefish, sturgeon, tench, roach, pike, pike-perch, sole, turbot, halibut, yellowtail, bass, bream, kingfish, milkfish, tilapia, tilapia, mullet, grouper, eels and aquarium fish such as goldfish, angel fish, clown fish, cichlids, corydoras, danio, discus, eel, gourami, guppy, loach, minnow, molly, platy, Plecostumas, rainbow and platy variatus, rasbora, shark, sword, tetra, botia, knife fish, lionfish, archer fish, flounder, golby, half beak, mono, needle fish, pipe fish, puffer, scat (green and red), rabbitfish, bumble bee, twin spot damsel, yellowtail damsel, barbed squirrel, wrasse, black-spotted puffer, trigger fish, puffer, and butterfly fish.

37. The method of claim 36, wherein, the animal is a fish selected from the group consisting of salmon, tuna, trout, sea bass, turbot, halibut, sea bream, kingfish, barramundi, grouper, carp, tilapia, and catfish.

38. The method claim 24, wherein the animal is a shellfish.

39. The method of claim 38, wherein the shellfish is selected from the group consisting of prawns, shrimps, lobsters, crayfishes, yabbies, crabs, abalone, mussels, oysters, cockles, sea urchins, sea cucumbers and polychaete worms.

40. The method of claim 24, wherein the production trait is growth.

41. The method of claim 24, wherein the production trait is fertility.

42. The method of claim 24, wherein the production trait is modulation of the gut microbiome.

43. The method claim 24, wherein, in an egg-laying animal, the production trait is egg production.
